(19) United States
(12) Patent Application Publication
(10) Pub. No.: US 2022/0005608 A1
(43) Pub. Date: Jan. 6, 2022

(54) METHOD OF PREDICTING DISEASE, GENE OR PROTEIN RELATED TO QUERIED ENTITY AND PREDICTION SYSTEM BUILT BY USING THE SAME

(71) Applicant: STANDIGM INC., Seoul (KR)

(72) Inventors: Hee Jung KOO, Gyeonggi-do (KR); Seokjin HAN, Seoul (KR); Chiwon SON, Gyeonggi-do (KR); Jang Ho LEE, Incheon (KR); Tae Yong KIM, Gyeonggi-do (KR); Chanung JEONG, Seoul (KR); Jinhan KIM, Seoul (KR); Sang Ok SONG, Gyeonggi-do (KR); So Jeong YUN, Gyeonggi-do (KR)

(21) Appl. No.: 17/297,352

(22) PCT Filed: Feb. 1, 2021
(86) PCT No.: PCT/KR2021/001299

§ 371 Date: May 26, 2021

(30) Foreign Application Priority Data
Jan. 31, 2020 (KR) 10-2020-0012169
Dec. 23, 2020 (KR) 10-2020-0182375

(51) Int. Cl. G16H 50/50; G06N 3/08

(52) U.S. Cl. CPC G16H 50/50; G06N 3/08

(57) ABSTRACT

Provided is a method, whereby data is collected from a plurality of databases to build a graph database, and an artificial neural network is trained based on the data stored in the built graph database so that an entity, for example, a disease, a gene, or a protein related to a queried entity on the artificial neural network for which the training has been completed, may be predicted, and a system built by using the same.

### TECHNICAL FIELD

The present invention relates to a method, whereby data is collected from a plurality of databases to build a graph database, and an artificial neural network model is trained based on the data stored in the built graph database so that an entity, for example, a disease, a gene, or a protein related to an entity queried on the artificial neural network for which the training has been completed, may be predicted, and a prediction system built by using the same.

### BACKGROUND ART

In the drug development process, identifying a drug target is one of the most important step in early stages, and a target that if modulated is most likely to have a therapeutic effect needs to be selected to increase the success rate in future clinical trials.

In order to reach the clinical trial stage of a new medicine, enormous manpower and costs are consumed, and it may be very significant to identify a target in the early stages in order to effectively find a cure for a disease.

In the related art, selection of the target is merely the collection of data from a plurality of databases to simply present a connection relation between previously disclosed data. Thus, there are many problems in selecting a new target for drug development beyond the collection of existing data.

The related prior art is as follows.

Korean Patent No. 10-2035658 discloses a system for recommending a drug repositioning candidate, in which drug and disease trait information and gene-related information are extracted from large-scale big data, such as literature information databases (DB) and genome information databases (DB), a drug-drug/disease-disease similarity matrix is built from the extracted drug and disease trait information and gene-related information, a drug-disease edge score based on literature information and a drug-disease edge score based on genomic information are computed according to the similarity matrix, and a final predicted score of the drug-disease edge is computed from the computed drug-disease edge scores so that drug repositioning candidates are recommended.

However, since the above-described system does not use an artificial neural network model, the accuracy of the recommendation is poor, and output information is different from the present invention. Also, since data is simply linearly integrated in the process of integrating data from a plurality of databases, it is difficult to check the relation between data with little correlation.

Korean Patent No. 10-1878924 discloses a method of predicting a candidate group for drug repositioning using a biological network, in which drugs, acting genes, and disease genes are associated with an activation/inhibition relation. In the biological network, when arbitrary drug information is input to the network, the shortest path between a drug and a disease gene is extracted, the correlation between the drug and the disease gene is quantified, and the computed value is output to simulate the effect of the drug on the disease gene so that a candidate group for drug repositioning can be selected.

However, since an artificial neural network model is not used in the above-described method, the accuracy of selection is poor, and output information is different from the present invention. Also, since data is simply linearly integrated in the process of integrating data from a plurality of databases, it is difficult to check the relation between data with little correlation.

Japanese Patent Laid-open Publication No. 2019-220149 discloses a system for generating a prioritized gene for a disease query. In the system, data including a rare disease, a gene, a phenotype for a rare disease, and a biological pathway are collected from a plurality of databases, estimated association is derived by applying Graph Convolution-based Association Scoring (GCAS), and the estimated association is added to a heterogeneous network to create a Heterogeneous Association Network for Rare Diseases (HANRD) so that prioritized genes for a disease query can be output.

It is similar to the present invention that the heterogeneous network includes several different types. However, nodes and edges are used with their types not classified even though several types have been collected from a plurality of databases and it is considered only whether or not there is a connection between nodes. Further, information about the entire vicinity of a node is used without a specific context or rationale and the artificial neural network model is not used, so that there is a disadvantage in that the accuracy of the result is low.

Accordingly, the present inventors have invented a system in which data collected from a plurality of databases are grouped and their types are defined based on their properties, and a database is built by reflecting the specified type, so that an entity related to a queried entity for an arbitrary entity (keyword) query, such as a disease, a gene, or a protein, can be presented with high accuracy.

### PRIOR ART DOCUMENTS

Korean Patent No. 10-2035658 (registered on Oct. 23, 2019)

Korean Patent No. 10-1878924 (registered on Jul. 17, 2018)

Japanese Patent Laid-open Publication No. 2019-220149 (published on Dec. 26, 2019)

### DISCLOSURE

### Technical Problem

The present invention provides a method and a system, whereby data related to diseases, genes, and compounds are collected, a graph database is built by using the collected data, nodes are embedded from the built database, and an artificial neural network is trained based on embedding results and high-importance paths so that a list of diseases, genes, or proteins may be output in the order of high relevance to an arbitrary entity query.

### Technical Solution

According to an aspect of the present invention, there is provided a prediction method including (a) defining disease-related in data included data collected from each of a plurality of databases as a first node, defining gene-related data included in the data as a second node, and defining compound-related data included in the data as a third node, performed by using a node definition module (131); (b) defining a relation between the first through third nodes defined by the node definition module 131 as an edge, performed by using an edge definition module (132); (c) defining a path wherein edges defined by the edge definition module (132) for each node pair are connected to each other, performed by using a path definition module (133); (d) computing scores of edges included in a path of the node pair according to a predetermined method so as to compute a path score for each node pair, performed by using a path score computation module (151); (e) extracting, for each preset path type (metapath), some of a plurality of paths included in the path type from node pair paths based on the path scores computed in (d), performed by using a path extraction module (152); (f) training an artificial neural network having a preset structure based on the paths extracted by the path extraction module (152) for each path type of a node pair and the first through third nodes, performed by using a data training module (160); (g) querying one keyword among a disease, a gene, and a compound, or keyword pair in the trained artificial neural network, performed by using an input module (170); and (h) outputting entities associated with the queried keyword or association of the queried keyword pair through computation of the artificial neural network, performed by using an output module (180).

The prediction method may further include, after the step (c) and before the step (d), performing real number vectorization so that a real number vector value is assigned to each of the first through third nodes defined by the node definition module (131) in a multi-dimensional space, performing real number vectorization so that a real number vector value is assigned to each edge type of the edge defined by the edge definition module (132) in the multi-dimensional space, so as to perform embedding on each of the first through third nodes and the edge types, performed by using an embedding module (140), wherein the step (d) further includes computing scores of edges included in a path of a node pair according to the predetermined method by using the real number vector values of the first through third nodes and the edge types embedded by the embedding module (140) and summing the computed scores of the edges so as to compute a path score for each node pair path, performed by using the path score computation module (151), and wherein the step (f) further includes training the artificial neural network having a preset structure based on the path extracted by the path extraction module (152) for each path type of a node pair and the first through third nodes embedded by the embedding module (140), performed by using the data training module (160).

The first node may include name data of a disease, anatomy data of a disease, and symptom data of a disease, and the second node may include name data of a gene, name data of a protein, gene ontology data of a gene, anatomy data of a gene, biological pathway data of a gene, and biological pathway data of a protein, and the third node may include name data of a compound, pharmacologic class data of a compound, and side effect data of a compound.

The edge definition module (132) may be configured to classify defined edges into one edge among a disease-gene relation edge, a gene-compound relation edge, a disease-compound relation edge, a gene-related edge, a disease-related edge, and a compound-related edge, the disease-gene related edge may include a gene-disease association edge type and a gene-disease regulation relation edge type, and the gene-compound relation edge may include a compound-gene binding relation edge type and a compound-gene regulation relation edge type, and the disease-compound relation edge may include a compound-disease treatment relation edge type, and the gene-related edge may include a gene-anatomy data regulation/expression relation edge type, a gene covariation relation edge type, a gene-gene ontology relation edge type, a gene-pathway relation edge type, a gene or protein interaction edge type, and a genetic interference-gene regulation relation edge type, the disease-related edge may include a disease-anatomy relation edge type, a disease-symptom relation edge type, and a disease co-occurrence similarity relation edge type, and the compound-related edge may include a compound-side effect relation edge type, a compound structural similarity relation edge type, and a compound-pharmacologic class relation edge type.

The prediction method may further include step (c) defining a path wherein the edges defined by the edge definition module (132) are connected to each other for each node pair, performed by using the path definition module (133), wherein the number of the edges in the pat is two or more and five or fewer.

The prediction method may further include step (c) defining a path wherein the edges defined by the edge definition module (132) are connected to each other for each node pair, performed by using the path definition module (133), wherein the number of the edges in the path is two or more and three or fewer.

The path type may be classified based on the combination of the number of edges constituting a path, the order of the edges, and types of the edges.

The prediction method may include step (e) extracting some of a plurality of paths included in a pre-determined path type for each node pair, performed by using the path extraction module (152), wherein some of the paths are extracted in an order of highest path scores computed in the step (d).

The prediction method may further include step (f) applying an attention mechanism for assigning different weights to the paths extracted by the path extraction module (152) according to nodes included in the paths and a path type to the artificial neural network.

The keyword pair may include one keyword among a disease, a gene, and a compound and another keyword that has a different type of keywords from the one keyword, and the step (h) may include outputting entities related to the keyword queried in the step (g) and outputting entities of different types from the queried keyword or association of the queried keyword pair.

The artificial neural network may be configured to score each of entities related to an arbitrary keyword to be queried according to a predetermined method, and the step (h) may further include outputting entities of different types from the queried keyword while being related to the arbitrary keyword to be queried in an order of highest sores through computation of the artificial neural network, performed by using the outputting module (180).

The prediction method may further include, after the step (h), when one entity among the entities output in the step (h) is selected, outputting one or more among an intermediate node, an edge, and a path from an arbitrary keyword to be queried to a selected entity in a form of a graph.

The prediction method may further include step (a) defining each of disease-related data, gene-related data, and compound-related data extracted by a natural language processing module (120) as first through third nodes, performed by using the node definition module (131), and the step (b) may further include defining a relation between the disease-related data, the gene-related data, and the compound-related data derived by the natural language processing module (120) as an edge, performed by using the edge definition module (132).

The prediction method may further include assigning a unique identifier (ID) to each of the first through third nodes defined by the node definition module (131), performed by using an ID assignment module (134), wherein the assigned ID is the same between an arbitrary term and a synonym or an abbreviation of the arbitrary term.

The prediction method may further include performing word embedding on each of the disease-related data, the gene-related data, and the compound-related data extracted by the natural language processing module (120) in a multi-dimensional space, performed by using the embedding module (140), wherein a distance between the disease-related data, the gene-related data, and the compound-related data is determined according to an extraction frequency of data pairs included in the data.

The prediction method may further include removing or inserting one or more nodes among nodes defined by the node definition module (131) or removing or inserting a new edge that is not defined by the edge definition module (132), wherein the artificial neural network is configured to output through an output layer different entities related to an arbitrary keyword to be queried through an input layer, so as to perform computation based on a dataset in which the one or more nodes are removed or inserted or the new edge is removed or inserted.

The prediction method may further include collecting user data including association of one or more arbitrary node pairs from a user database, performed by using a data collection module (110), wherein the artificial neural network is configured to perform computation based on a dataset on which the user data is reflected.

The prediction method may further include, based on a specific time point at which data is collected from each of the plurality of databases, collecting data disclosed in the plurality of databases after the specific time point, extracting disease-related data, gene-related data, and compound-related data included in the data collected after the specific time point and deriving a relation between the extracted disease-related data, gene-related data, and compound-related data, performed by using the natural language processing module (120), querying an arbitrary keyword in the artificial neural network, performed by using the input module (170), outputting entities related to the arbitrary keyword to be queried, performed by using the output module (180), and verifying whether or not a first data pair has association based on whether the first data pair comprising the queried keyword and the output entity is included in a second data pair connected to each other with the relation derived through the natural language processing module (120).

According to another aspect of the present invention, there is provided a system built by using the prediction method described above.

According to another aspect of the present invention, there is provided a program stored in a computer-readable recording medium to execute the prediction method described above.

### Advantageous Effects

According to the present invention, genes or proteins that may be drug targets for specific disease can be predicted with high accuracy by using a machine learning algorithm.

Since genes or proteins related to a queried disease are predicted according to the machine learning algorithm, it is possible to identify new target genes or proteins that are not known.

In addition, the association between the queried disease and the predicted genes or proteins is illustrated and output so that the basis for prediction can be presented.

Since each of a node, an edge, and a path that constitute a graph database is grouped according to its property and a score thereof is evaluated according to the property, the efficiency of using a heterogeneous network that uses a variety of types of networks is maximized.

Since graph database embedding and word embedding may be performed together, computation of the similarity between diseases, the similarity between genes, and the similarity between compounds can be performed.

Instead of training the artificial neural network model based on the collected data, embedded nodes and paths with high importance are used for training so that computation throughput can be reduced and a computation time can be minimized.

Since data collected from a user database through a corresponding user account access, as well as data collected from a plurality of data bases, can be used in prediction, a corresponding researcher can obtain the result of the prediction in which his/her own research context is reflected.

In addition, since computation can be further performed in a situation where insertion or removal of one or more of a node, an edge, and a path is reflected according to user input, the result of prediction in a kind of virtual experiment environment can be obtained.

### DESCRIPTION OF DRAWINGS

FIG. 1 is a block diagram of a system according to an embodiment;

FIGS. 2 and 3 are schematic views of a method of building a system according to an embodiment;

FIG. 4 is a view for describing nodes and edges used in a process of building a system according to the present invention;

FIG. 5 is a view for describing a training process of a data training module used in the process of building a system according to the present invention;

FIG. 6 is a view showing a result of outputting a gene or a protein related to a disease queried through an output module in the order of highest scores when a certain disease is queried in a system according to the present invention;

FIG. 7 is a view showing a result of schematically outputting a path between a gene or a protein selected from among the genes or the proteins output in FIG. 6 and a queried disease;

FIG. 8 is a view for describing an implementation so that manipulation can be applied to an existing graph database by a user command;

FIG. 9 is a view for describing a browsing function implemented in a system built according to the present invention;

FIG. 10 is a view for describing a state in which the relation between arbitrary node pairs is output in the form of a graph in a system built according to the present invention;

FIG. 11 is a view showing results of a verification experiment for verifying the excellence of a system built according to the present invention; and

FIG. 12 is a flowchart illustrating a method according to an embodiment.

### MODE FOR INVENTION

Hereinafter, the present invention will be described in detail with reference to the accompanying drawings.

Hereinafter, the term “node pair” refers to data including pairs of nodes defined by a node definition module. Specifically, the node pair may be data including a pair of different types of nodes, and a first node-second node pair, a first node-third node pair, and a second node-third node pair are concepts that may be included in the node pair.

Hereinafter, the term “keyword” is different from the above-described node, and refers to entities, words or symbols that can be input by an input module, and may include names of diseases, names of genes, names of proteins, and names of compounds. Similarly, “keyword pair” refers to data including pairs of keywords, and refers to data including different types of keywords (disease-gene, disease-protein, disease-compound, gene-compound, protein-compound, etc.).

Hereinafter, the term “gene” refers to an individual unit of genetic information including a specific sequence in a genome including DNA or RNA, and also includes individual units of genetic information including a specific amino acid sequence in a genome including protein as well as DNA and RNA.

1. Description of System and Method

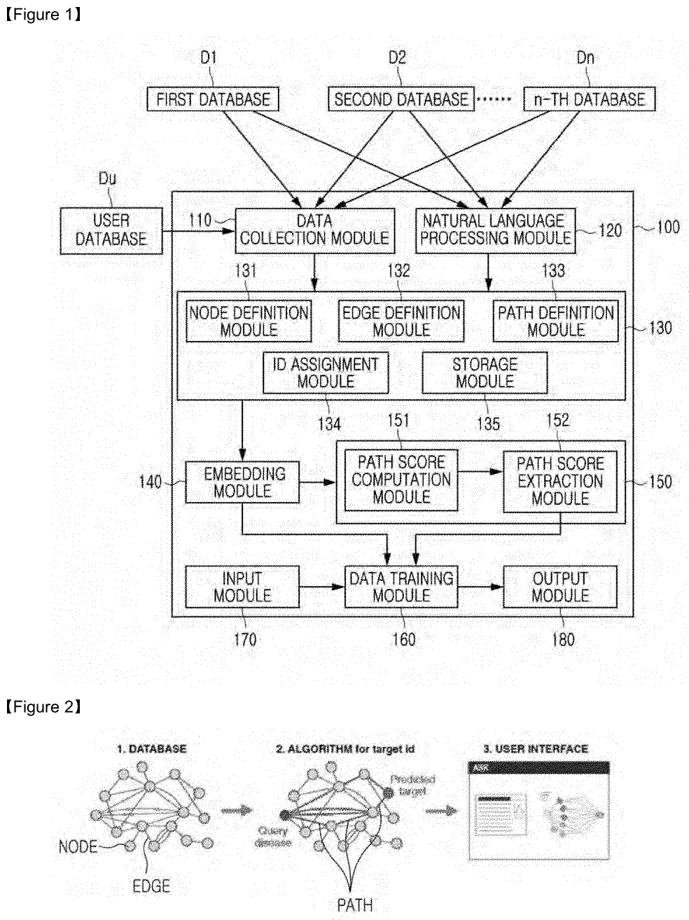

Referring to FIG. 1, a system according to an embodiment of the present invention may include a data collection module 110, a natural language processing module 120, a definition module 130, an embedding module 140, a preprocessing module 150, a data training module 160, an input module 170, and an output module 180.

The data collection module 110 is configured to collect data from a plurality of databases D1, D2, . . . , and Dn. The data collected by the data collection module 110 may be, for example, gene expression data, compound-protein binding data, data obtained by itemizing information described in dissertations, document data, etc. However, the data of the present invention is not limited to the above-described form. The format of the data is not limited as long as the data includes disease-related data, gene-related data or compound-related data.

To this end, the system according to the embodiment of the present invention may be connected to communication with a plurality of databases D1, D2, . . . , and Dn, and the plurality of databases D1, D2, . . . , and Dn may be public databases. However, the database of the present invention is not limited thereto, and the plurality of databases D1, D2, . . . , and Dn may be a private database and may include a dissertation database, a medical information database, a pharmaceutical information database, and a search portal database.

The data collection module 110 may collect first data related to a disease, second data related to a gene, and third data related to a compound from each of the plurality of databases D1, D2, . . . , and Dn.

The first data is data related to a disease and may include name data of diseases, anatomy data of diseases (for example, anatomical data of the body where a disease occurs, and in the case of liver cancer, the liver may be the case), and symptom data of diseases. In other words, the first data includes not only the term referring to a disease itself, but also all of the terms necessary to provide information related to the disease.

The second data is data related to a gene and may include name data of genes, gene ontology data of genes, anatomical data of genes (for example, information on the body tissue in which a gene is expressed, and when genes highly expressed in the liver are preferentially considered in order to find a gene associated with liver cancer, the liver may be the case), and biological pathway data of genes. The gene ontology data may include biological process data of a gene, cellular component data of a gene, and molecular function data of a gene. In other words, the gene ontology data is a concept that includes not only the term referring to a gene itself, but also all of the terms necessary to provide information related to the gene.

The anatomical data may be included in the first data or the second data. For example, when the data includes information that gene A is expressed in tissue B, the tissue B may be collected as the second data, which is gene-related data. And, when the data includes information that disease C occurs in tissue D, the tissue D may be collected as the first data, which is disease-related data.

The third data is compound-related data and may include name data of a compounds, pharmacologic class data of compounds, and side effect data of compounds. In other words, the third data is a concept that includes not only the term referring to a compound itself, but also all of the terms necessary to provide information related to the compound.

However, the present invention is not limited to the above types, and it will be understood that any data related to diseases, genes, and compounds and any data necessary for predicting the relation between diseases, genes, and proteins, may be included.

The natural language processing module 120 is configured to extract entities from a text included in the document data collected by the data collection module 110, and thereby deriving the relation between the entities through a preset natural language processing algorithm.

The entity extracted and the relation of the entities derived by the natural language processing module 120 may be defined as a node and an edge, respectively, and a detailed description thereof will be provided below.

That is, the natural language processing module 120 is configured to recognize and extract a disease-related term contained in document data as a first entity, a gene-related term as a second entity, a compound-related term as a third entity, and a term describing relation between the first to third entities as a fourth entity, respectively.

In addition, the natural language processing module 120 is configured to derive relations between the first to fourth entities by using the extracted first to fourth entities through a predetermined method.

Extracting the first through fourth entities and deriving relations between the entities by using the natural language processing module 120 according to the present invention may be performed using a pre-trained neural network model. That is, the neural network model may be configured to be trained based on training data labeled with each of the first through fourth entities, to extract the first through fourth entities from document data to be queried, and to derive the relation between the entities.

According to the related art, after a term to be extracted is pre-stored in an index dictionary, only the pre-stored term is extracted from a text. In this case, when terms that are not previously stored in the index dictionary are included in the text, the terms cannot be extracted, and eventually, a system can be built only within the known scope of knowledge.

However, according to the present invention, the neural network model is trained based on the training data labeled, for example, as to which part of a text corresponds to which entity among the first through fourth entities, not extracting the terms stored in the index dictionary, so that, even for terms that have not been defined in advance, it is possible to extract entities in consideration of the form of the term itself, the context, etc. Thus, it is possible to extract entities from new categories as well as categories known in existing papers and to derive the relation between the entities.

The definition module 130 defines nodes and edges, which are components of a graph database, further defines a path, and includes a node definition module 131, an edge definition module 132, and a path definition module 133.

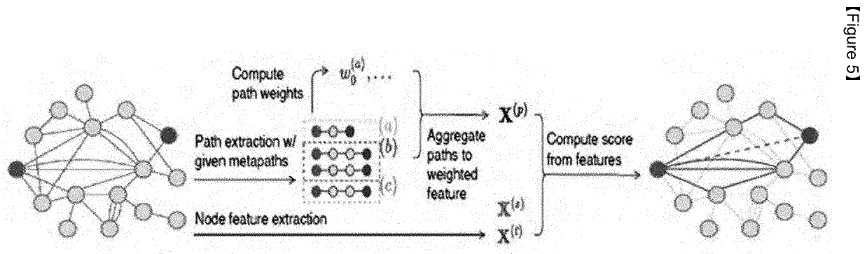

The node definition module 131 may group the first data in the data collected by the data collection module 110 into name data of a disease, anatomical data of a disease, symptom data of a disease, etc., may group the collected second data into name data of a gene, biological process data of a gene, anatomical data of a gene, cellular component data of a gene, molecular function data of a gene, biological pathway data of a gene, etc., and may group the collected third data into name data of a compound, pharmacologic class data of a compound, and side effect data of a compound, thereby classifying the types of 11 groups (see FIG. 4). However, the present invention is not limited to the above number, and various types of groups may be added.

In another embodiment, the node definition module 131 may group each of the first entity, the second entity, and the third entity extracted through the natural language processing module 120 according to a predetermined method and may define each of the first entity, the second entity, and the third entity as a node.

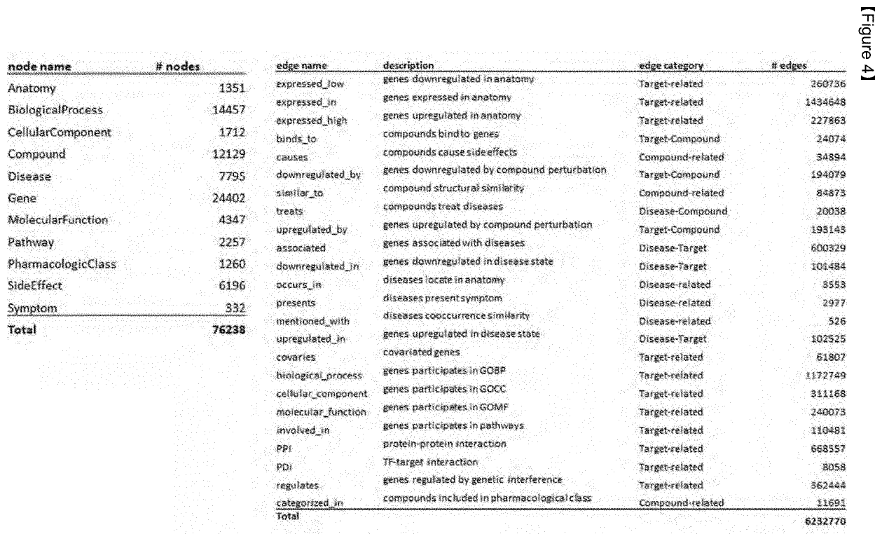

In other words, the node definition module 131 may define the first through third entities extracted by the natural language processing module 120 and the first through third data collected from a plurality of databases, respectively, as first through third nodes (see FIG. 3). As will be described below, the edge definition module 132 defines the relation between the first through third entities derived by the natural language processing module 120 and the relation between the first through third data as an edge. Examples of nodes defined according to the present invention and edges connecting the nodes are shown in FIG. 3.

In addition, the node definition module 131 defines data included in the grouped data as each node according to the type.

That is, the node definition module 131 defines the first data (entity) as each node for each kind, defines the second data (entity) as each node for each kind, and defines the third data (entity) as each node for each kind.

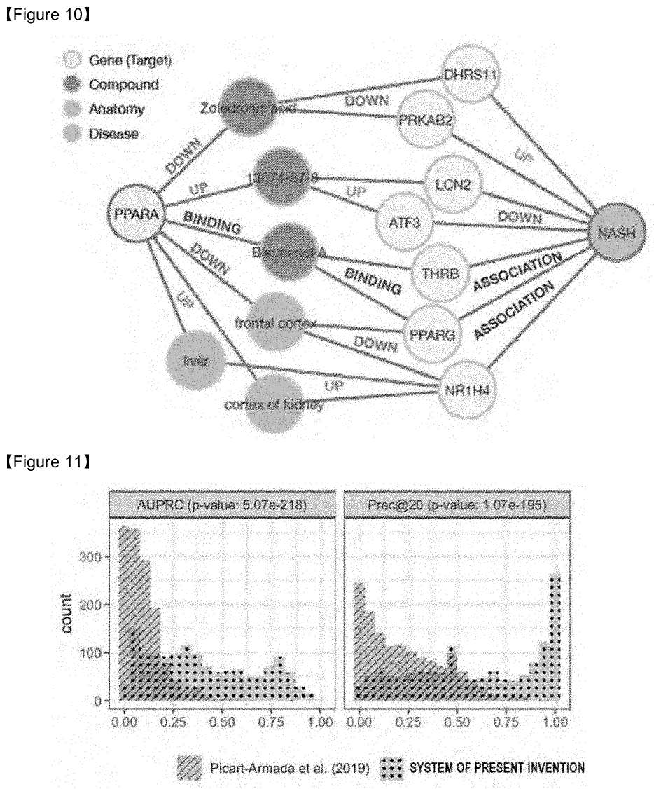

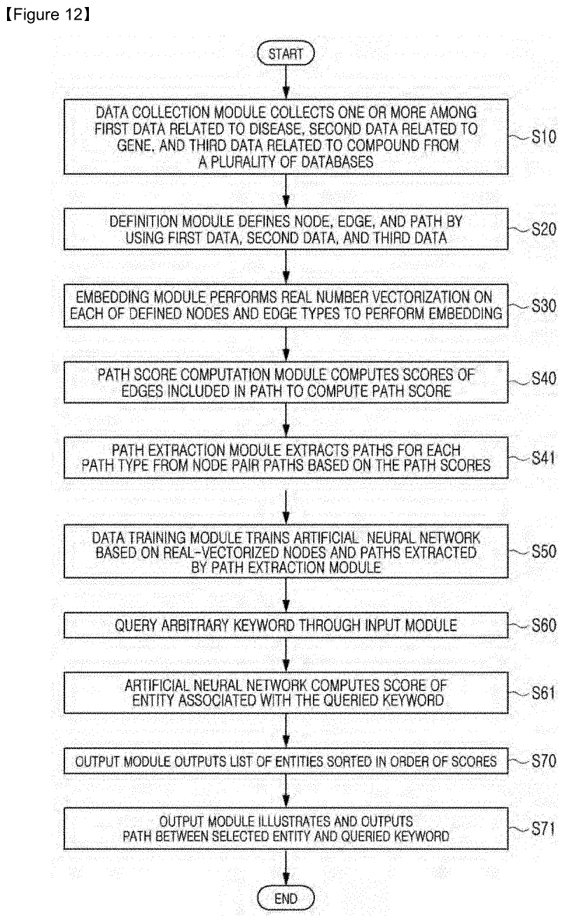

In FIG. 10, nodes defined by the node definition module 131 are illustrated, and more specifically, gene-related nodes of PPARA, DHRS11, PRKAB2, LCN2, ATF3, THRB, PPARG, and NR1H4, compound-related nodes of zoledronic acid, 13674-87-8, and Bisphenol A, anatomical data-related nodes of a disease or a gene of frontal cortex, liver, and cortex of kidney, and disease-related nodes of NASH are shown.

The edge definition module 132 defines the relation between nodes defined by the node definition module 131 as an edge.

The edge refers to a connection relation between one node and another node, and the edge definition module 132 defines the relation between the nodes included in the collected data as an edge connecting the corresponding node pair to each other.

For example, when one document data includes the text “Breast cancer patients may present with lump symptoms, and can be treated by using tamoxifen hormone compounds”, one edge for connecting the node “breast cancer” and the node “lump” may be defined, and one edge connecting the node “breast cancer” and the node “tamoxifen hormone compounds” may be defined.

As such, the edge definition module 132 may define the relation between nodes as an edge by using the first data, the second data, and the third data collected by the data collection module 110, and may group the defined edges, as grouping in the node definition module 131.

FIGS. 3 and 4 illustrate edges that are defined, grouped, and typed by the edge definition module 132.

Referring to FIG. 3, the edges defined by the edge definition module 132 may be classified into a disease-gene relation edge Disease-Target, a gene-compound relation edge Target-Compound, a disease-compound relation edge Disease-Compound, a gene-related edge Target-related, a disease-related edge Disease-related, and a compound-related edge Compound-related.

FIG. 4 shows an edge type (metaedge) in which each edge is typed.

Specifically, the disease-gene relation edge Disease-Target includes a gene-disease association edge type (e.g., “associated”) and a gene-disease regulation relation edge type (e.g., “downregulated_in” and “upregulated_in”).

The gene-compound relation edge Target-Compound includes a compound-gene binding relation edge type (e.g., “binds_to”) and a compound-gene regulation relation edge type (e.g., “downregulated_by” and “upregulated_by”).

The disease-compound relation edge Disease-Compound includes a compound-disease treatment relation edge type (e.g., “treats”).

The gene-related edge Target-related includes a gene-anatomy regulation/expression relation edge type (e.g., “expressed_low,” “expressed_in,” and “expressed_high”), a gene covariation relation edge type (e.g., “covaries”), a gene-gene ontology relation edge type (e.g., “biological_process,” “cellular component,” and “molecular function”), a gene-pathway relation edge type (e.g., “involved_in”), a gene or protein interaction edge type (e.g., “PPI” and “PDI”), and a genetic interference-gene regulation relation edge type (e.g., “regulates”).

The disease-related edge Disease-related includes a disease-anatomy relation edge type (e.g., “occurs_in”), a disease-symptom relation edge type (e.g., “presents”), and a disease co-occurrence similarity relation edge type (e.g., “mentioned_with”).

The compound-related edge Compound-related includes a compound-side effect relation edge type (e.g., “causes”), a compound structural similarity relation edge type (e.g. “similar_to”), and a compound-pharmacologic class relation edge type (e.g. “categorized_in”).

That is, the edge definition module 132 may classify the edges into 24 groups. However, it will be understood that the present invention is not limited to the above-described number, and various types of groups may be added.

The path definition module 133 defines a path that includes one or more, specifically two or more edges defined by the edge definition module 132, and the included edges are connected to each other.

More specifically, the path definition module 133 defines a path wherein edges defined by the edge definition module 132 for each node pair are connected to each other.

More specifically, a node pair wherein nodes are connected to each other by two or more and five or fewer edges may be defined as a path, and more specifically, a node pair wherein nodes are connected to each other by two or more and three or fewer edges may be defined as a path. A node pair connected by four or more edges may be excluded from a valid path because when nodes are connected to each other through too many edges, the association between the nodes may be considered low.

Referring to FIG. 10, one path of PPARA-zoledronic acid-DHRS11-NASH is shown, and one path of PPARA-liver-NR1H4-NASH is shown.

For the paths defined by the path definition module 133, path types may be determined according to combinations of the number of edges constituting a path, the order of the edges, and types of the edges shown in FIG. 4.

For example, the path “AKT1-associates-Alzheimer's disease-resembles-Parkinson's disease” has the path type such as “Gene-associates-Disease-resembles-Disease”. In other words, the path including A (type a) edge-B (type b) edge may be defined as the path type (a,b), and the path including A (type a) edge-B (type b) edge-C (type c) edge may be defined as the path type (a,b,c). These path types may be treated as different types from each other.

In addition, the path definition module 133 may classify some of the types of a plurality of paths into a preset path type (metapath). As will be described below, path types that do not correspond to the preset path types are excluded from a training process according to the present invention.

For example, the path definition module 133 may set a path type including an edge type in the sequence of Disease-mentioned_with-Disease-associates_with-Gene as a preset path type among a plurality of path types. In another example, the path definition module 133 may set a path type including an edge type in the sequence of Disease-treated_by-Compound-binds_to-Gene-interacts_with-Gene as a preset path type. The present invention is not particularly limited thereto, a preset path type may be set by a system administrator. The efficiency and accuracy of training may be improved by training with only meaningful paths among paths connecting an arbitrary node pair.

In addition, the path definition module 133 may exclude a path type including an edge type in the sequence of Disease-treated_by-Compound-downregulates-Gene-regulated_by-Gene and a path type including an edge type in the sequence of Disease-downregulates-Gene-upregulated_by-Compound-binds_to-Gene among a number of path types determined according to the combination of the number of edges, the order of the edges, and the types of the edges from a preset path type. Also, path types that are excluded from preset path types may be defined by a system administrator. By excluding paths that are meaningless or less significant from paths that connect an arbitrary node pair in the training process, the efficiency of training and the accuracy of computation may be improved.

The ID assignment module 134 is configured to assign a unique ID to each of the nodes defined by the node definition module 131.

That is, the ID assignment module 134 according to the present invention assigns a unique ID to an arbitrary term representing each node, and terms that may be determined to be the same as the arbitrary term, such as synonyms and abbreviations of the arbitrary term, are assigned with the same ID as the arbitrary term.

Meanwhile, there may be a case where two or more IDs are assigned to an arbitrary term. For example, since alpha-fetoprotein is referred to as an abbreviation of AFP, and an ID of 174 may be assigned to both alpha-fetoprotein and AFP. And, an ID of 7726, which is an ID of TRIM26, may be assigned to AFP because AFP is a synonym for the gene TRIM26. That is, two IDs, 174 and 7726, may be assigned to AFP. In this case, the ID assignment module 134 assigns, to AFP, the ID of 174 that matches the full name of AFP (alpha-fetoprotein) rather than the ID of 7726.

A unique ID is mapped for each node and stored in the storage module 135, and the ID assignment module 134 assigns a unique ID to each node by using the IDs stored in the storage module 135.

The embedding module 140 performs embedding one or more among the nodes defined by the node definition module 131, the edges and the edge type (metaedge) defined by the edge definition module 132, and the paths and the preset path type (metapath) defined by the path definition module 133.

More specifically, the embedding module 140 performs embedding each of the nodes defined by the node definition module 131 and the edge types defined by the edge definition module 132.

Hereinafter, an example of an embedding method using the embedding module 140 will be described.

First, the embedding module 140 initializes all nodes defined by the node definition module 131 into k-dimensional random vectors. Here, k may be 128. However, the present invention is not limited thereto, and it is possible to initialize nodes into vectors of real numbers composed of various random variables such as 64, 256, 512, 1024, etc.

Next, the embedding module 140 initializes all edge types defined by the edge definition module 132 into k dimensional random vectors. Here, k may be 128. However, the present invention is not limited thereto, and it is possible to initialize all edge types into vectors of real numbers composed of various random variables such as 64, 256, 512, 1024, etc.

Next, it is determined whether an arbitrary node pair is connected to each other by an edge having an edge type defined by the edge definition module 132, and the result of determination is entered into supervised learning labeled data. When an arbitrary node pair (source node, target node) is connected by an edge having an edge type defined by the edge definition module 132, a datum of 1 will be entered, and when the arbitrary node pair (source node, target node) is not connected by an edge having an edge type defined by the edge definition module 132, a datum of 0 will be entered.

A k-dimensional vector is adjusted so that a prediction function that takes three k-dimensional vectors (source node, target node, and edge type) as inputs matches whether or not it is actually connected. Here, the prediction function may be a model such as TransE, HoIE, or DistMult, but the present invention is not limited thereto, and various prediction function models may be applied to the present invention.

When the adjustment is completed, k-dimensional real number vectors corresponding to each node are computed as the result of embedding of the corresponding node and the edge type.

In addition to the above-described methods, various embedding methods may be performed. As a result of embedding by the embedding module 140, each node may be mapped to a single point in a k-dimensional space. As a result of embedding by the embedding module 140, each of the first through third nodes may be mapped in the k-dimensional space, and edge types may be also embedded in the k-dimensional space.

The embedding module 140 may perform word embedding of the first through third entities extracted by the natural language processing module 120.

When word embedding is performed by the embedding module 140, each entity is mapped on a multi-dimensional space, and a distance between entities may be determined based on the frequency at which a corresponding entity-pair is expressed in document data.

That is, when there are 100 document data describing the relation between a disease A and a gene B, and there are 10 document data describing the relation between the disease A and a gene C, entities may be mapped in multi-dimensional space so that a distance between A and B is closer than a distance between A and C.

Information such as association between each entity, for example, association between a disease and a gene, association or similarity between genes, association or similarity between diseases, and association or similarity between compounds, may be further obtained by computing the distance between the entities.

The preprocessing module 150 may include a path score computation module 151 that computes the score of a path by computing scores of edges included in the path according to a predetermined method, and a path extraction module 152 that extracts some paths of paths defined by the path definition module 133 based on the score of a path computed by the path score computation module 151.

A method of computing the score of a path by computing the scores of the edges included in the path by the path score computation module 151 will be described.

The scores of each edge included in a path are computed by using the respective nodes and edge types embedded by the embedding module 140. Specifically, an edge included in the path has a k-dimensional real number vector (map) of a corresponding edge type and a k-dimensional real number vector of the start and end nodes of the edge, and the score of the edge may be computed from these real number vectors. As an example of a specific computation method, the prediction function used in the embedding module 140 may be applied, and similarity of mapped nodes may also be applied.

In the computation method based on the similarity of the mapped nodes, the higher the similarity between the nodes mapped in the k-dimensional space, the higher the score assigned to the edges connecting the nodes. As the similarity computation method, a method of computing an angle between one vector and another vector (more specifically, a method of computing a cosine value of two vectors) may be applied, and various methods that compute the degree of similarity between vectors may be applied.

The score of a path including n edges (n is an integer greater than or equal to 1) may be computed by summing the edge scores of each of n edges. In the case of a path including n+1 edges, the score of the path may be computed by summing the scores of each of the n+1 edges.

The path extraction module 152 extracts some paths for each preset path type (metapath).

As described above, the path type may be classified according to the number of edges included in a path, the order of the edges, and types of the edges. For example, a path including an edge A (type a)-edge B (type b) may be defined as having the path type (a,b), and a path including an edge A (type a)-edge B (type b)-edge C (type c) may be defined as having the path type (a,b,c). These paths may be treated as having different path types to each other.

More specifically, for paths having a preset path type among paths of an arbitrary node pair, some paths may be extracted for each path type in the order of the highest score by using the path score computed by the path score computation module 151. In an example, five paths may be extracted for each path type. However, it will be understood that the present invention is not limited thereto, and fewer than 5 or more than 5 paths may be extracted.

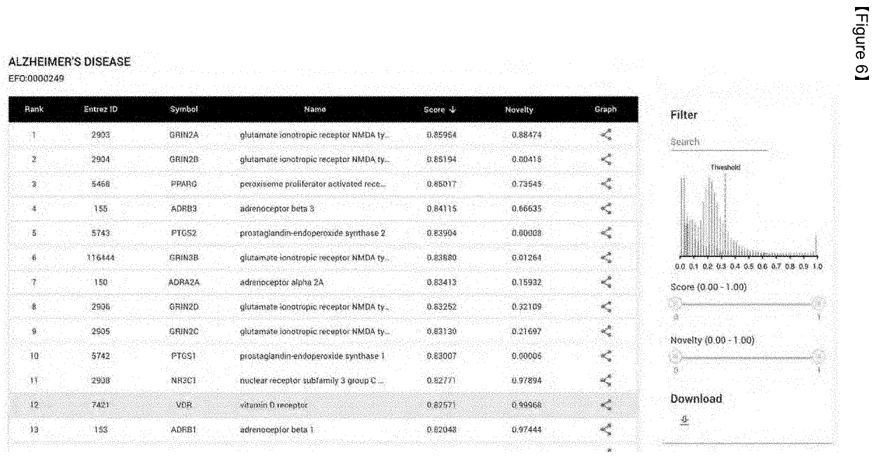

The data training module 160 may train an artificial neural network model with the embedding result performed by the embedding module 140 and the path extracted by the path extraction module 152, and may apply an attention mechanism and a hyperparameter optimization mechanism to the trained artificial neural network model. Here, the attention mechanism may be a method of assigning different weights to the paths extracted by the path extraction module 152 based on all nodes included in the extracted paths and the path type of the extracted paths. For example, the data training module 160 trains the artificial neural network model based on node features mapped in the k-dimensional space and path features having the high importance, i.e., a path assigned a higher weight among paths for connecting an arbitrary node pair (see FIG. 5). In addition, it is possible to train the artificial neural network model with whether there is association between already-known entities.

Computational efficiency is improved by training embedding results of grouped data and paths with high importance only, rather than training the entire data collected from the plurality of databases.

Here, the artificial neural network may be Deep Neural Network (DNN), Convolutional Neural Network (CNN), Deep Convolutional Neural Network (DCNN), Recurrent Neural Network (RNN), Restricted Boltzmann Machine (RBM), Deep Belief Network (DBN), Single Shot Detector (SSD), Multi-layer Perceptron (MLP), or a model based on an attention mechanism, but the present invention is not limited thereto, and various artificial neural network models may be applied to the present invention.

When training of the artificial neural network model is completed by the above process, the artificial neural network model may output entities related to a keyword that is queried in an input layer through an output layer. Specifically, entities that are related to an arbitrary keyword to be queried and have different types from the queried keyword may be output in the order of the highest score (that is, when a disease is queried, genes, proteins, or compounds are output). Thus, it is possible to grasp the entities with high importance, which are highly associated with the keyword being queried.

The input module 170 may have a form of an input device, and may be, for example, a touch panel or a keyboard, but the present invention is not particularly limited as long as the input module 170 receives a user command and transmits the command to the system according to the present invention.

In addition, the output module 180 has a form of an output device, and may be, for example, a monitor or a display panel, but the present invention is not particularly limited as long as a computation result of the system according to the present invention can be visually checked by the output module 180.

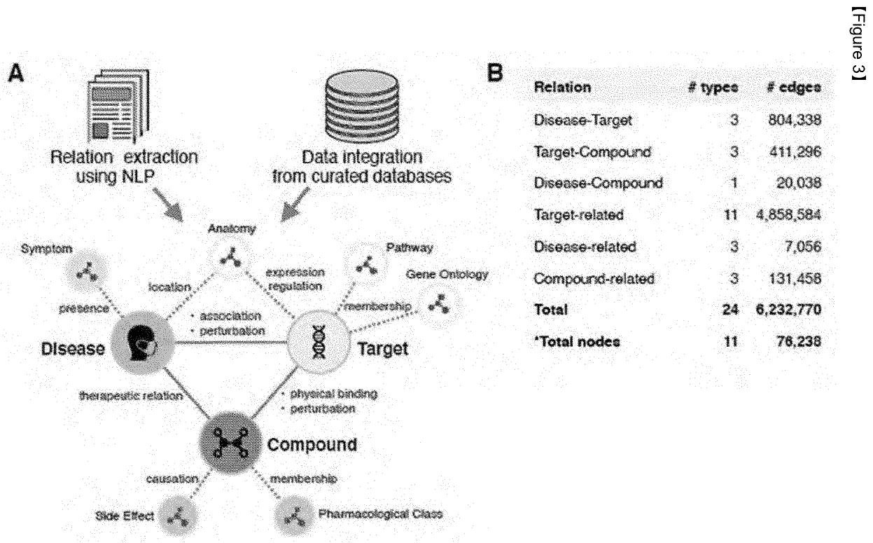

When system construction according to the present invention is completed, a keyword (e.g., arbitrary disease, gene, protein, or compound, etc.) or keyword pair (disease-gene, disease-compound, gene-compound, etc.) input through the input module 170 may be queried to the data training module 160, that is, the artificial neural network model. And, entities associated with the queried keyword may be output in the order of highest importance through the output module 180, or whether or not the keyword pair being queried is associated with each other may be output through computation of the artificial neural network model (see FIG. 2).

FIG. 6 illustrates a state in which a disease called ALZHEIMER'S DISEASE is queried through the input module 170 in the artificial neural network model and a result of computation is output through the output module 180. GRIN2A, GRIN2B, PPARG, ADRB3, PTGS2, etc. as entities associated with ALZHEIMER'S DISEASE are output with listing in the order of highest significance.

In the present invention, the symbol of an entity related to the keyword to be queried is output, and further the specific name of the entity, how the relation between the keyword and the entity is novel in light of known knowledge (Novelty), and a score quantified by the algorithm for the degree of association between the keyword and the entity are also output. Based on this, the user may select an arbitrary entity (e.g., a gene or a protein) from the output list.

In addition, results that satisfy a specific score scope and specific Novelty condition may be output by user selection. For example, when setting to output only entities with a score of 0.8 or more and Novelty of 0.9 or more, only a list of entities satisfying the condition may be displayed.

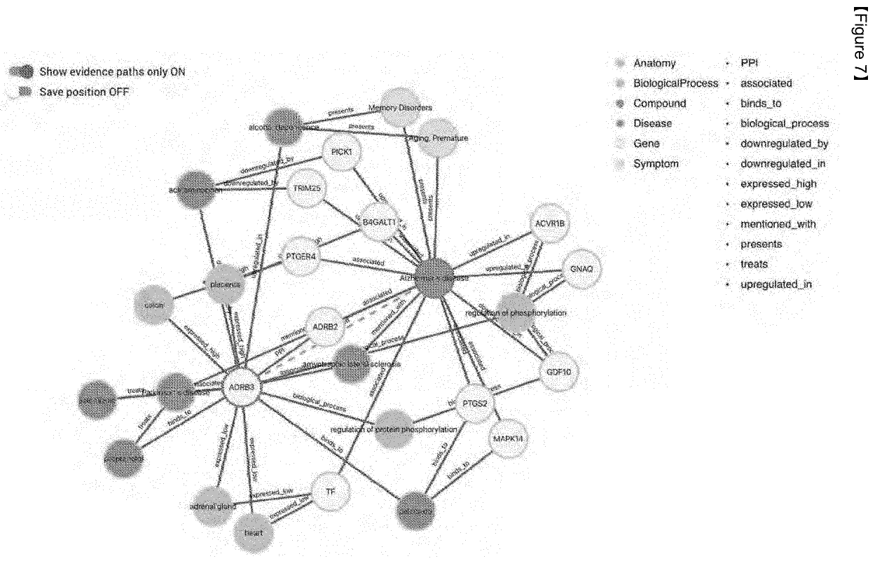

When an arbitrary entity is selected through the input module 170, a graph-type chart composed of nodes and edges between the queried keyword and the selected entity may be output (see FIG. 7). By generating a chart in the form of a graph, the result is more intuitive than simply listing related nodes. In addition, nodes included in a path between the queried keyword and the selected entity, and edges may all be output, so it is possible to visualize and show the rationale of the prediction.

For example, when an arbitrary disease is queried through the input module 170, genes or proteins are output with sorting in the order of high degree of association with the disease, and paths between the selected gene or protein and the input disease are visualized and output, thereby helping researchers develop new compounds that target the genes or proteins.

In addition, when an arbitrary gene or protein is queried through the input module 170, diseases are output with sorting in order of high degree of association with the gene or protein, and paths between the selected disease and the input gene or protein are visualized and output, thereby enabling researchers to query bidirectionally.

According to the present invention, when a specific disease is queried, genes or proteins related to the disease are output with sorting in order of importance, and when an arbitrary gene or protein is queried, diseases are output with sorting in the order of importance related to the gene or protein and output. Thus, research on the development of target genes/proteins and compounds for a specific disease, and studies to predict related diseases with a specific gene or protein or a specific compound may all be performed on one system, providing comprehensive information and convenience to researchers. In addition, since bidirectional cross-validation is possible, the accuracy of prediction is further improved.

Meanwhile, in the present invention, when an arbitrary keyword pair is queried, an artificial neural network model may be used that is configured to compute a score predicting the degree of association between keywords in the keyword pair.

It has been described above that in the artificial neural network model, any one of a disease, a gene, a protein, and a compound may be queried, and in another example, a keyword pair may be queried.

Entities of different types from the queried keyword while being associated with the queried keyword are output (i.e., when a disease called Alzheimer's disease is queried, genes, proteins, and compounds related to Alzheimer's disease are output) through computation of the artificial neural network model.

FIG. 6 shows a state in which different entities related to Alzheimer's disease are output when the keyword Alzheimer's disease is queried.

Here, a score is also displayed on each output entity together, and the displayed score is computed from the artificial neural network model.

The artificial neural network model computes the score of an entity based on the association and importance of the “queried keyword”-“predicted entity”. In other words, the artificial neural network model searches a path belonging to a preset path type (metapath) among the possible paths connecting “queried keyword”-“predicted entity” (e.g., disease-target), and compute a weight by determining the degree of association with “queried keyword”-“predicted entity” for each path. For example, a path having high association with “queried keyword”-“predicted entity” may be assigned with a high weight, and a path not related to “queried keyword”-“predicted entity” may be assigned with a low weight.

Next, based on the computed weight, several paths are merged into one real number vector.

Next, a score may be computed using multi-layer perceptron (MLP) that takes a merged real number vector, embedding of the queried keyword and embedding of the predicted entity as inputs.

The score output from the artificial neural network model corresponds to a score indicating the likelihood that the queried keyword-predicted entity node pair is actually related to each other. For example, the higher the score shown in FIG. 6 is, the higher the probability is of a gene or a protein being related to the disease ALZHEIMER'S DISEASE.

The training in the artificial neural network model used in the present invention may be performed in the manner described below. As the data for training, (i) for each preset path type (metapath), some paths selected based on the path score among a plurality of paths corresponding to the preset path type and (ii) a first node to a third node are used.

More specifically, (i) for each preset path type, some paths in the order of the highest path score and (ii) the real number vector values, which are the results of embedding a first node to a third node defined by the node definition module 131 and edge types defined by the edge definition module 132 are used as data for training. Additionally, (iii) an attention mechanism for assigning different weights to paths based on the nodes included in the paths being trained and the path type of the paths being trained may be applied.

The artificial neural network model, by training the training data, can output entities associated with an arbitrary keyword being input into an input layer of the artificial neural network model through the input module 170 and the degree of importance with regard to the relation between the entities and the arbitrary keyword.

That is, the artificial neural network model is allowed to train the training data described above, so as to compute the degree of importance between the arbitrary keyword being input into the input layer and the entities being output through the output layer.

FIG. 6 shows an image in which the score of the degree of importance of relation between ALZHEIMER'S DISEASE and different types of entities through the computation by the artificial neural network model.

The system according to the embodiment of the present invention may further collect data from a user database Du.

“User database Du” refers to a database in which a dataset obtained through an experiment by a user of the system is stored.

Data from the user database Du may be further added to the built graph database, which is built by collecting data from the plurality of databases D1, D2, . . . , and Dn by the data collection module 110. This may include data verified by experiments, etc., for example, data that shows the relation between a pair of disease and protein, thereby obtaining a prediction result reflecting the research context.

Since the user database Du stores private data, it may be configured to collect data from the user database Du only by accessing the system with an account matching the user of the user database Du.

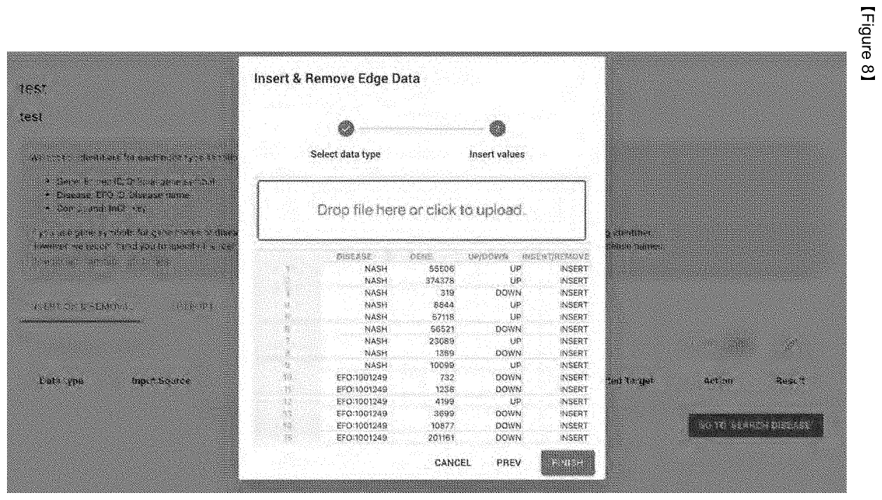

According to the present invention, manipulation by a user command through the input module 170 may be performed on a graph database built by using data collected from an existing public database (see FIG. 8).

According to an embodiment of the present invention, manipulation, such as insertion of information on the change in expression of a gene (increased or decreased expression) when a specific disease occurs, insertion of information on the change in expression of a gene (increased or decreased expression) when a specific compound is administered, insertion of information on a protein that binds to a specific compound, and insertion or removal of specific gene nodes may be performed. In addition, since computation of the artificial neural network model according to the present invention is performed based on the data on which the manipulation is reflected, it is possible to check the effect of modification applied by the user on the result.

Preferably, the manipulation may be performed in a category different from the content of data presented in the existing public databases D1, D2, . . . , and Dn. It is because, for example, if it is assumed that the information that the probability of developing disease B increases when the expression of gene A is increased is already published, the existing graph database will not be modified even if the manipulation according to the above is performed. On the other hand, when a new category of data is added that is not a category of the data presented in the existing public database (e.g., when it is not known from the existing data that compound C inhibits the expression of gene A and the corresponding content is added), the existing graph database may be modified. Through the above manipulation, it is possible to compare the result of the existing database and the result of the modified database that has been manipulated by the user, and accordingly, it is possible to check how much the manipulation applied by the user has affected the result.

For example, a command of performing computation after inserting or removing an arbitrary node may be input through the input module 170. In addition, a command to perform computation after inserting or removing an edge or a path may be input. That is, computation by the artificial neural network model may be performed assuming that a node desired by the system user exists additionally or does not exist. As an example, when a command to perform computation after removing the “CHD1” node is input through the input module 170, the artificial neural network model may perform computation in a situation in which the CHD1 node the edges corresponding to the relation between the CHD1 node and other nodes have been removed. In other words, assuming that “CHD1” is knocked out, genes or proteins associated with the queried disease may be output. When a node is removed through user manipulation, an edge connecting the removed node and another node may also be removed.

When the command to perform computation after inserting a node desired by the system user is input, on the contrary, computation may be performed in a situation where the inserted node and a relation between the inserted node and an arbitrary node are inserted, and a result in a virtual environment that occurs as data desired by the user is inserted or removed, may be obtained.

The result information that is obtained according to the above manipulation may be separately stored in the user database Du of each user, and the user database Du is accessible only to the user, so that security may also be maintained.

The system according to the present invention may be provided with a search function as well as a query command. That is, when a search word to be searched is input, a database browsing function in which data including the input search word is output may be provided.

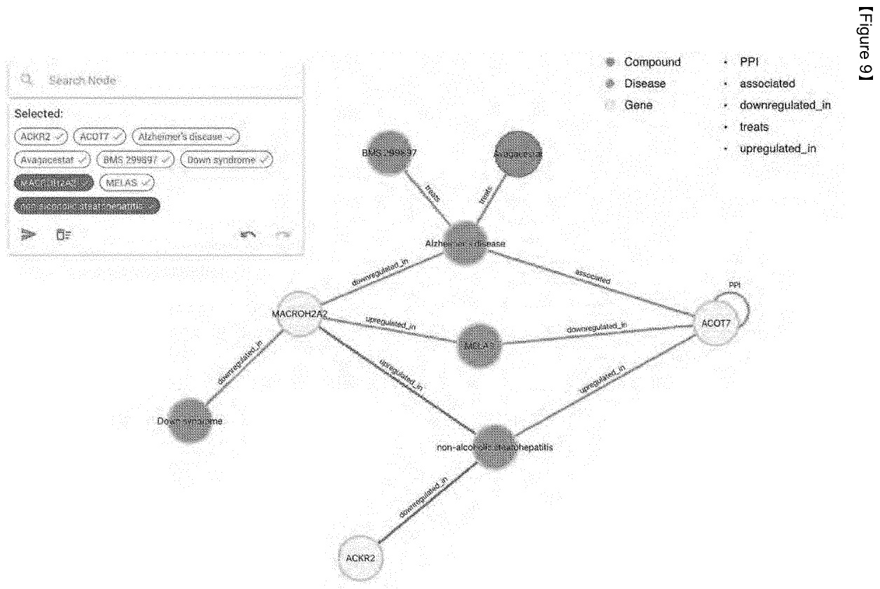

In other words, it is configured to search for additional information about the predicted results and components of important paths generated as a result of the query command, and obtain not only the data including the queried search word but also expanded information connected to the search word (see FIG. 9).

In addition, one of the database built according to the present invention and the customized database (database built by collecting more user data from a user database or database built by reflecting user manipulation) is selected, and various nodes and edges in the selected database may be searched to obtain the necessary information.

When an arbitrary keyword is queried, lists of entities (e.g., target genes or proteins) related to the queried keyword (e.g., disease) are output. When selecting any one of the entities in the lists, a search function may be also provided in the queried keyword-entity path graph. That is, the user may freely search for nodes and edges related to a specific node on the graph as shown in FIG. 9.

The system according to the present invention is equipped with a verification function so that it is possible to verify performance.

After building a system according to the present invention by collecting data stored in the plurality of public databases D1, D2, . . . , and Dn up to a specific time point, document data updated since the specific time point in the plurality of public databases D1, D2, . . . , and Dn are collected to derive the relation between the entities from the document data through the natural language processing module 120.

In addition, a node pair (first data pair) predicted with a certain threshold or higher reliability among the node pairs predicted according to the present invention is included in an entity pair (second data pair) extracted through the natural language processing module 120, it is possible to cross-verify that the corresponding node pair is actually relevant.

2. Verification Experiment

A verification experiment was conducted to verify the excellence of a system built according to the present invention.

First, a list of diseases to be evaluated was selected. Here, a disease to be evaluated refers to a disease in which a specific gene or protein is already known to be related to the disease and which can thus be checked whether the known specific gene or protein is predicted to have a high score from the list of predicted results (genes or proteins) when the corresponding disease is queried in the system of the present invention.

For the results output by querying the disease to be evaluated, two indicators were computed: 1) AUPRC, and 2) Prec@20 (Precision @ top 20).

FIG. 11 shows, when the same set of diseases is queried, the distribution of AURPC and Prec@20 index values in a conventional Random Forest Model configured to predict factors associated with the queried disease and in the system built according to the present invention.

As the value of the x-axis is closer to 1, it can be seen that the system has a higher prediction performance, and it is proved through this experiment that the system built according to the present invention has superior prediction performance compared to the conventional prediction model.

All or at least a part of the configuration of the system according to the embodiment of the present invention may be implemented in the form of a hardware module or a software module, or a combination of a hardware module and a software module.

Here, the software module may be understood as, for example, an instruction executed by a processor that controls computation in the system, and such an instruction may have a form mounted in a memory in the disease-related factor prediction system.

While the present invention has been particularly shown and described with reference to exemplary embodiments thereof, it will be understood by those of ordinary skill in the art that various changes in form and details may be made therein without departing from the spirit and scope of the present invention as defined by the following claims.

### DESCRIPTION OF REFERENCE NUMERALS

100: system
110: data collection module
120: natural language processing module
130: definition module
131: node definition module
132: edge definition module
133: path definition module
134: ID assignment module
135: storage module
140: embedding module
150: preprocessing module
151: path score computation module
152: path extraction module
160: data training module
170: input module
180: output module

1. A prediction method, which comprises:
(a) defining disease-related data included in data collected from each of a plurality of databases as a first node, defining gene-related data included in the data as a second node, and defining compound-related data included in the data as a third node, performed by using a node definition module;
(b) defining a relation between the first through third nodes defined by the node definition module as an edge, and grouping the defined edges into one or more edge types (metaedges) according to their respective properties, performed by using an edge definition module;
(c) defining a path that edges defined by the edge definition module for each node pair are connected to each other, performed by using a path definition module;
(d) embedding the first through third nodes defined by the node definition module and the edge types defined by the edge definition module, performed by using an embedding module;
(e) computing an edge score of each of the edges included in a path by using the embedding results of the edge types performed in the step (d) and the embedding results of the first through third nodes, and computing a path score for each path by using edge scores of all of the edges included in a path of an arbitrary node pair, performed by using a path score computation module, as a step for computing a path score for each path defined in the step (c);
(f) extracting, for each preset path type (metapath), some of a plurality of paths included in the preset path type from arbitrary node pair paths in the order of the highest to the lowest of the path scores computed in the step (e), performed by using a path extraction module;
(g) in order to allow for an artificial neural network having a preset structure to compute and output the association between the keywords being queried in an input layer and the entities to be predicted or the association between the keyword pairs being input, training the artificial neural network on the embedding results performed in the step (d) and the pathway extracted by the path extraction module for each preset path type of a node pair among the pathways of arbitrary node pairs performed in the step (f), performed by using a data training module, wherein the pathways not extracted by the path extraction module are excluded from the training;
(h) querying one keyword among a disease, a gene, and a compound, or keyword pair in the trained artificial neural network, performed by using an input module; and
(i) outputting entities associated with the queried keyword or association of the queried keyword pair through computation of the artificial neural network, performed by using an output module,
wherein path types are classified according to combinations of the number of edges, the order of the edges, and types of the edges constituting a path, and the preset path types are at least part of the classified types.

2. The prediction method of claim 1, wherein the step (d) further comprises performing real number vectorization so that a real number vector value is assigned to each of the first through third nodes defined by the node definition module in a multi-dimensional space, and performing real number vectorization so that a real number vector value is assigned to each edge type of the edge defined by the edge definition module in the multi-dimensional space, so as to perform embedding on each of the first through third nodes and each of the edge types, performed by using the embedding module, and
wherein the step (e) further comprises computing scores of edges included in a path of a node pair according to the predetermined method by using the real number vector values of the first through third nodes and the edge types embedded by the embedding module and summing the computed scores of the edges so as to compute a path score for each node pair path, performed by using the path score computation module.

3. The prediction method of claim 1, wherein the first node comprises name data of a disease, anatomy data of a disease, and symptom data of a disease; the second node comprises name data of a gene, name data of a protein, gene ontology data of a gene, anatomy data of a gene, biological pathway data of a gene, and biological pathway data of a protein; the third node comprises name data of a compound, pharmacologic class data of a compound, and side effect data of a compound.

4. The prediction method of claim 1, wherein the edge definition module is configured to classify defined edges into one edge among a disease-gene relation edge, a gene-compound relation edge, a disease-compound relation edge, a gene-related edge, a disease-related edge, and a compound-related edge,
the disease-gene relation edge comprises a gene-disease association edge type and a gene-disease regulation relation edge type,
the gene-compound relation edge comprises a compound-gene binding relation edge type and a compound-gene regulation relation edge type,
the disease-compound relation edge comprises a compound-disease treatment relation edge type,
the gene-related edge comprises a gene-anatomy regulation/expression relation edge type, a gene covariation relation edge type, a gene-gene ontology relation edge type, a gene-pathway relation edge type, a gene or protein interaction edge type, and a genetic interference-gene regulation relation edge type,
the disease-related edge comprises a disease-anatomy relation edge type, a disease-symptom relation edge type, and a disease co-occurrence similarity relation edge type, and
the compound-related edge comprises a compound-side effect relation edge type, a compound structural similarity relation edge type, and a compound-pharmacologic class relation edge type.

5. The prediction method of claim 1, wherein the step (c) further comprises defining a path that the edges defined by the edge definition module are connected to each other for each node pair, performed by using the path definition module, wherein the number of the edges in the path is two or more and five or fewer.

6. The prediction method of claim 5, wherein the step (c) further comprises defining a path that the edges defined by the edge definition module are connected to each other for each node pair, performed by using the path definition module, wherein the number of the edges in the path is two or more and three or fewer.

7. (canceled)

8. (canceled)

9. The prediction method of claim 1, wherein the step (g) further comprises applying an attention mechanism for assigning different weights to the paths extracted by the path extraction module according to nodes included in the paths and a path type to the artificial neural network.

10. The prediction method of claim 1, wherein the keyword pair comprises one keyword among a disease, a gene, and a compound and another keyword that has a different type of keywords from the one keyword, and the step (i) comprises outputting entities related to the keyword queried in the step (h) and outputting entities of different types from the queried keyword or association of the queried keyword pair.

11. The prediction method of claim 1, wherein the artificial neural network is configured to score each of entities related to an arbitrary keyword to be queried according to a predetermined method, and the step (i) further comprises outputting entities of different types from the queried keyword while being related to the arbitrary keyword to be queried in an order of highest sores through computation of the artificial neural network, performed by using the outputting module.

12. The prediction method of claim 1, further comprising, after the step (i),
(j) when one entity among the entities output in the step (i) is selected, outputting one or more among an intermediate node, an edge, and a path from an arbitrary keyword to be queried to a selected entity in a form of a graph.

13. The prediction method of claim 1, wherein the step (a) further comprises defining each of disease-related data, gene-related data, and compound-related data extracted by a natural language processing module as first through third nodes, performed by using the node definition module, and the step (b) further comprises defining a relation between the disease-related data, the gene-related data, and the compound-related data derived by the natural language processing module-as an edge, performed by using the edge definition module.

14. The prediction method of claim 1, further comprising assigning a unique identifier (ID) to each of the first through third nodes defined by the node definition module, performed by using an ID assignment module, wherein the assigned ID is the same between an arbitrary term and a synonym or an abbreviation of the arbitrary term.

15. The prediction method of claim 13, further comprising performing word embedding on each of the disease-related data, the gene-related data, and the compound-related data extracted by the natural language processing module in a multi-dimensional space, performed by using the embedding module, wherein a distance between the disease-related data, the gene-related data, and the compound-related data is determined according to an extraction frequency of data pairs included in the data.

16. The prediction method of claim 1, further comprising removing or inserting one or more nodes among nodes defined by the node definition module or removing or inserting a new edge that is not defined by the edge definition module, wherein the artificial neural network is configured to output through an output layer different entities related to an arbitrary keyword to be queried through an input layer, so as to perform computation based on a dataset in which the one or more nodes are removed or inserted or the new edge is removed or inserted.

17. The prediction method of claim 1, further comprising collecting user data including association of one or more arbitrary node pairs from a user database, performed by using a data collection module, wherein the artificial neural network is configured to perform computation based on a dataset in which the user data is reflected.

18. The prediction method of claim 1, further comprising:
based on a specific time point at which data is collected from each of the plurality of databases, collecting data disclosed in the plurality of databases after the specific time point;
extracting disease-related data, gene-related data, and compound-related data included in the data collected after the specific time point and deriving a relation between the extracted disease-related data, gene-related data, and compound-related data, performed by using the natural language processing module;
querying an arbitrary keyword in the artificial neural network, performed by using the input module;
outputting entities related to the arbitrary keyword to be queried, performed by using the output module; and
verifying whether or not a first data pair has association based on whether the first data pair comprising the queried keyword and the output entity is included in a second data pair connected to each other with the relation derived through the natural language processing module.

19. A system built by using the prediction method of claim 1.

20. A computer program stored in a computer-readable recording medium to execute the prediction method of claim 1.
